(19) United States
(12) Patent Application Publication
(10) Pub. No.: US 2009/0054373 A1
(43) Pub. Date: Feb. 26, 2009

(54) CHITIN DERIVATIVES FOR HYPERLIPIDEMIA

(75) Inventors: Andre Aube, St-Sauveur (CA); Ryszard Brzezinski, Sherbrooke (CA); Gilles Dupuis, Sherbrooke (CA); Jean-Guy Lehoux, Sherbrooke (CA)

Correspondence Address: KNOBBE MARTENS OLSON & BEAR LLP, 2040 MAIN STREET, FOURTEENTH FLOOR, IRVINE, CA, 92614, US

(73) Assignee: DNP Canada Inc., Montreal (Quebec) (CA)

(21) Appl. No.: 12/097,754

(22) PCT Filed: Jul. 21, 2006
(86) PCT No.: PCT/CA2006/001213

§ 371 Date: Sep. 15, 2008

Related U.S. Application Data
(60) Provisional application No. 60/750,827, filed on Dec. 16, 2005.

(51) Int. Cl. A61K 31/722; A61P 9/10

(52) U.S. Cl. 514/55

(57) ABSTRACT

The preferred embodiments relate to chitin derivatives for prevention or treatment of hyperlipidemia, such as hypercholesterolemia and the resultant atherosclerosis in a mammal. The preferred embodiments are useful for reducing serum cholesterol, and/or cholesteryl ester, triglycerides, phospholipids and fatty acids in a mammal.

### BACKGROUND OF THE INVENTION

1. Field of the Invention

The present invention relates to the field of therapeutic agents useful in lowering cholesterol (particularly low-density cholesterol) and/or, cholesteryl esters, triglycerides, phospholipids, and fatty acids in a mammal, such as a human. More particularly, the invention relates to compositions comprising chitin derivatives.

2. Description of the Related Art

It is well known that hyperlipidemic conditions associated with elevated concentrations of total cholesterol and low-density lipoprotein (LDL) cholesterol are major risk factors for cardiovascular disease, such as atherosclerosis. Numerous studies have demonstrated that a low plasma concentration of high density lipoprotein (HDL) cholesterol (good cholesterol) is a powerful risk factor for the development of atherosclerosis (Barter and Rye, Atherosclerosis, 121, 1-12 (1996). HDL is one of the major classes of lipoproteins that function in the transport of lipids through the blood. The major lipids found associated with HDL include cholesterol, cholesteryl esters, triglycerides, phospholipids, and fatty acids. The other classes of lipoproteins found in the blood are low-density lipoprotein (LDL), intermediate density lipoprotein (IDL), and very low-density lipoprotein (VLDL). Since low levels of HDL cholesterol increase the risk of atherosclerosis, methods for elevating plasma HDL cholesterol would be therapeutically beneficial for the treatment of cardiovascular diseases, such as atherosclerosis. Cardiovascular diseases include, but are not limited to, coronary heart disease, peripheral vascular disease, and stroke.

One therapeutic approach to hyperlipidemic conditions has been the reduction of total cholesterol. Known use is made of the understanding that HMG CoA reductase catalyzes the rate-limiting step in the biosynthesis of cholesterol (The Pharmacological Basis of Therapeutics, 9th ed., J. G. Hardman and L. E. Limberd, ed., McGraw-Hill, Inc., New York, pp. 884-888 (1996)). HMG CoA reductase inhibitors (including the class of therapeutics commonly called “statins”) reduce blood serum levels of LDL cholesterol by competitive inhibition of this biosynthetic step (M. S. Brown, et al., J. Biol. Chem. 253, 1121-28 (1978)). Several statins have been developed or commercialized throughout the world. Atorvastatin calcium sold in North America under the brand Lipitor® is a potent reductase inhibitor. It is described in European Patent 409,281.

Warnings of side effects from use of HMG CoA reductase inhibitors include liver dysfunction, skeletal muscle myopathy, rhabdomyolysis, and acute renal failure. Some of these effects are exacerbated when HMG CoA reductase inhibitors are taken in greater doses. For example, a patient treated with 10 mg/day of Lipitor® may notice mild side effects. These side effects may greatly increase by simply raising the daily dose to 20 mg/day.

Furthermore, it has been shown that patients with well-controlled lipid profiles when treated with 10 mg/day of Lipitor® or another low dose statin may experience a return to elevated lipid profiles and require a dosage increase.

It is also known in the art that natural chitosan derived from chitin may have cholesterol-lowering properties. Jing et al. disclose the cholesterol-lowering effects of natural chitosan having a molecular weight of 27 kDa and a degree of deacetylation of 89%. It was observed that the total serum cholesterol and lipoprotein levels of the patients were significantly reduced (Jing et al., J. Pharm. Pharmacol. 1997, 49: 721-723).

Ylitalo et al. teach that a natural chitosan having a molecular weight of 8 kDa is more effective in lowering cholesterol in rats than chitosan with a molecular weight of 2 or 220 kDa. Furthermore, it was also disclosed that preparations having natural chitosan with a molecular weight of 5 to 120 kDa seem to be the most efficient in lowering cholesterol (Ylitalo et al. Arzneim.-Forsh. Drug Res. 52, No. 1, 1-7 (2002). However, it should be noted that lower molecular weight chitosans are more expensive to produce due to different factors related to the process of manufacture. For instance, a higher amount of enzyme is needed to produce a lower molecular weight chitosan. It should also be noted that the chitosan used in the art is the natural glucosamine polymer obtained by deacetylation of chitin. However, such polymer has several drawbacks such as a reduced shelf life; a poor solubility in a physiological acidic environment such as the gastric milieu of the stomach.

Thus, although there are a variety of hypercholesterolemia therapies, there is a continuing need and a continuing search in this field of art for improved therapies.

### SUMMARY OF THE INVENTION

The preferred embodiments improve efforts for preventing and/or treating hyperlipidemia, such as by reducing serum cholesterol, by providing a composition comprising chitin derivatives.

An embodiment provides a pharmaceutical composition comprising chitin derivative.

An embodiment provides a method for the prevention or treatment of hyperlipidemia or hyperlipidemia-associated condition comprising administering a pharmaceutical composition comprising chitin derivative having a molecular weight of at least 10 kDa to about 240 kDa.

The chitosan derivative of the preferred embodiments is advantageously stable as compared to the natural polysaccharide chitosan and thus may be advantageously used in a pharmaceutical/nutraceutical composition for a prolonged period and thus prolong the shelf life of the latter. Natural polysaccharide chitosan remains stable for a period of a few weeks whereas the chitosan derivatives of the preferred embodiments will remain stable in the composition for at least 2 years.

The chitosan derivative composition of the preferred embodiments further has the advantage of significantly lowering the time and cost of manufacture all the while increasing the total yield of production.

### DETAILED DESCRIPTION OF THE PREFERRED EMBODIMENT

As used herein, the word “a” or “an”, when used in conjunction with the term “comprising” in the claims and/or the specification may mean “one” but it is also consistent with the meaning of “one or more”, “at least one” and “one or more than one”.

As used herein, the term “about” is used to designate a possible variation of up to 10%. Therefore, a variation of 1, 2, 3, 4, 5, 6, 7, 8, 9 and 10% of a value is included in the term “about”.

Finally, as used in the specification and claims, the words “comprising”, “having”, “including” or “containing” are inclusive or open-ended and do not exclude additional unrecited elements or method steps.

It has been found that chitin derivatives as defined herein below as a chitosan salt formed from any chitosan molecule associated with a negatively charged anion, having a molecular weight of at least 10 kDa can lower cholesterol levels. In a preferred embodiment, the chitin derivative has a molecular weight of from at least 10 kDa to about 120 kDa. In a further preferred embodiment, the chitin derivative has a molecular weight of from about 30 to about 90 kDa. In another preferred embodiment, the chitin derivative has a molecular weight of from about 40 to about 70 kDa.

Chitin is a polymer of β-1-4-N-acetyl-D-glucosamine. Chitin, an amino cellulose derivative, is the second most abundant polymer occurring in nature. A common source of chitin can be found in the cell walls of fungi, bovine cartilage, and the hard shells of insects and crustaceans. Waste from industrial microbiological plants using fermentation methods with fungal organisms is another source of chitin. Wastes from the shrimp, lobster, and crab seafood industries can contain about 10-30% chitin.

While there exists many extraction methods of the chitin from the crustacean shells, the principles of chitin extraction are relatively simple. In a certain treatment, the proteins are removed in a dilute solution of sodium hydroxide (such as about 1-10%) at high temperature (such as about 85-100° C.). Shells are then demineralized to remove calcium carbonate. This can be done by treating in a dilute solution of hydrochloric acid (1-10%) at room temperature. Depending on the severity of these treatments such as temperature, duration, concentration of the chemicals, concentration and size of the crushed shells, the physico-chemical characteristics of the extracted chitin can vary. For instance, three characteristics of the chitin, such as the degree of polymerization, acetylation, and purity, can be affected. Shell also contains lipids and pigments. Therefore, a decolorizing step is sometimes needed to obtain a white chitin. This can be done by soaking in organic solvents or in a very dilute solution of sodium hypochlorite. Again, these treatments can influence the characteristics of the chitin molecule.

Chitin can be deacetylated partially or totally. Such a deacetylated polymer is called chitosan. Chitosan compounds in a range of up to and exceeding 1×10⁶ kDa molecular weight are derived commercially from chitin. In nature, chitosan is present in cell walls of Zygomycetes, a group of phytopathogenic fungi. Because of its significant content of free amino groups, chitosan has a markedly cationic character and has a positive charge at most pHs. Short chain chitosans can be produced by a process disclosed in Canadian Patent 2,085,292, the disclosure of which is incorporated herein by reference.

As used herein, “chitin” refers to a polymer formed primarily of repeating units of β (1-4) 2-acetamido-2-deoxy-D-glucose (or N-acetylglucosamine). Not every unit of naturally occurring chitin is acetylated, with about 16% deacetylation.

As used herein, “chitosan” refers to chitin that has been partially or fully deacetylated. Chitosan is a polysaccharide formed primarily of repeating units of β (1-4) 2-amino-2-deoxy-D-glucose (or D-glucosamine). Further deacetylation of chitosan can be achieved by processing of chitin. Deacetylation values can vary with chitin sources and with processing methods.

As used herein, “derivative” refers to a chemical composition derived from another substance either directly or by modification or partial substitution.

Since chitin and chitosan are derivatives of each other, the terms “chitin derivative” and “chitosan derivative” can be used interchangeably and can encompass each other herein. Accordingly, the term “chitin derivative” is understood herein to encompass chitin, chitosan, and their derivatives.

As used herein, the terms “chitin derivative” and “chitosan derivative” can be used interchangeably and can encompass each other herein. The term “chitin derivative” is also understood herein to encompass a chitosan salt formed from any chitosan molecule associated with a negatively charged anion. A series of anions has been used for that purpose. For example, anions can be derived from inorganic acids. Preferred inorganic anions include, but are not limited to, sulfuric acid (sulfate), phosphoric acid (phosphate), hydrochloric acid (chloride), hydrobromic acid, hydroiodic acid, nitric acid, chloric acid, perchloric acid, boric acid, carbonic acid, hydrofluoric acid, pyrophosphoric acid and thiosulfate. Anion can also be derived from organic acids. Preferred organic anions include, but are not limited to, malic acid (malate), tartaric acid (tartrate), citric acid (citrate), lactic acid (lactate), succinic acid (succinate), acetic acid, benzoic acid, butyric acid, formic acid, methanethiol, propionic acid, pyruvic acid, valeric acid, mandellic acid, adipic acid, alginic acid, boric acid, carbonic acid, carminic acid, cyclamic acid, erythorbin acid, fumaric acid, gluconic acid, glutamic acid, guanylic acid, hydrochloric acid, inosinic acid, metatartaric acid, nicotinic acid, oxalic acid, pectic acid, phosphoric acid, sorbic acid, stearic acid, sulfuric acid, tannic acid and amino acids (e.g. aspartate and glutamate). Polymeric organic and inorganic anions are also useful for forming the chitosan salt, such as polyaspartate. Antioxydants including but not limited to ascorbic acid, citric acid, erythorbic acid and tartric acid may also be used to form the chitosan salt as they prevent oxidative degeneration of the cation (chitosan) salt.

Chitin derivatives can be produced by the process described in Canadian Patent 2,085,292, and recovered from solution using the process described in WO 2005/066213-A1, where the chitosan is salted out with a salting-out salt such as sulfates, phosphates, citrates, nitrates, malates, tartrates, succinates, propionates, lactates and hydrogen phosphates. More preferably, these salting-out salts may be organic or inorganic and may be selected from the group consisting of: ammonium or sodium sulfate; sodium or potassium phosphates; sodium or potassium citrate; sodium tartrate; sodium malate; sodium nitrate; sodium lactate; sodium malonate; sodium succinate; sodium acetate; sodium propionate. Thus, the preferred embodiments include any chitosan derivative obtained by any of the above-mentioned salts.

As an example, the citrate salt of chitosan can be illustrated as follows:

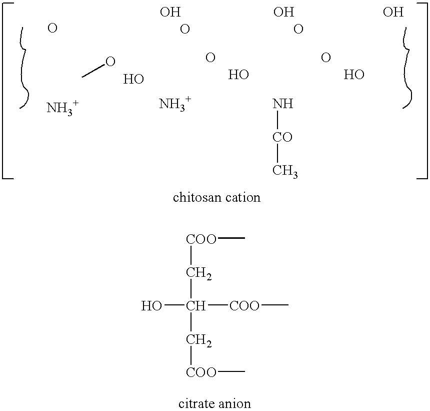

An approach for addressing hyperlipidemia is the use of chitin derivatives.

As used herein, “nutraceuticals” is understood to encompass any ordinary food that has components or ingredients added to give a specific medical or physiological benefit other than a purely nutritional effect. It is also understood to include functional foods, dietary supplements and over the counter products sold without a prescription.

As used herein, “functional foods” is understood to encompass any food consumed as part of a usual diet that is similar in appearance to, or may be, a conventional food, and is demonstrated to have physiological benefits and/or reduce the risk of chronic disease beyond basic nutritional functions.

In a mechanism of action, chitin derivatives, in particular, chitosan, can contain free amine groups which can attach themselves to lipids, such as cholesterol, and biliary acids via ionic bonds while in the intestinal tractus, forming an indissociable complex which is eventually excreted. Chitin derivatives therefore can prevent lipids, such as cholesterol, from ever entering the bloodstream and biliary acids from being reabsorbed and adding to the total cholesterol content. Also, in reaction, the liver eliminates more cholesterol by producing and secreting biliary acids into the intestines. Therefore, there is elimination of both food cholesterol and that of biliary acids rich in cholesterol.

### Molecular Weight

Chitin derivatives have many potential applications depending on their molecular weight. The molecular weight can be measured by any of a number of well-known techniques, including, without limitation, by SDS-PAGE or mass spectrometry. These techniques can yield various types of molecular weights, including without limitation, apparent molecular weight, a weight average molecular weight, or a number average molecular weight. An average high molecular weight chitin derivative is about 650 kDa. Some applications are typical of medium or low molecular weight chitin derivatives, ranging typically about 2-500 kDa. These applications include its use as an antifungal agent; a seed coating for improving crop yield; an elicitor of anti-pathogenic natural reactions in plants; a hypocholesterolemic agent in animals; an accelerator of lactic acid bacteria breeding; and a moisture-retaining agent for lotions, hair tonics and other cosmetics.

The molecular weight of chitin derivatives is a feature that is particular to a certain application. The molecular weight of the native chitin has been reported to be as high as many million Daltons. However, chemical treatment tends to bring down the molecular weight of the chitin derivative, ranging from 100 KDa to 1500 KDa. Further treatment of the chitin derivative can lower the molecular weight even more. Low molecular weight could be produced by different ways including enzymatic or chemical methods. Molecular weight of the chitin derivative can be measured by analytical methods, such as gel permeation chromatography, light scattering, or viscometry. Because of simplicity, viscometry is the most commonly used method.

In the preferred embodiments, the chitin derivative has a molecular weight of at least 10 kDa. Preferably, the chitin derivative has a molecular weight ranging from at least 10 kDa to about 240 kDa.

In another preferred embodiment, the chitin derivative has a molecular weight ranging from about 20 kDa to about 100 kDa.

In another embodiment, the chitin derivative preferably has a molecular weight of about 30 to about 80 kDa.

In another embodiment, the chitin derivative preferably has a molecular weight of about 40 to about 70 kDa.

Preferably, the chitin derivative has a molecular weight listed in Table 1.

TABLE 1
molecular weight of chitin derivative of preferred embodiments

| | | | | | | | | | |
|---|---|---|---|---|---|---|---|---|---|
| 10 | 11 | 12 | 13 | 14 | 15 | 16 | 17 | 18 | 19 |
| 20 | 21 | 22 | 23 | 24 | 25 | 26 | 27 | 28 | 29 |
| 30 | 31 | 32 | 33 | 34 | 35 | 36 | 37 | 38 | 39 |
| 40 | 41 | 42 | 43 | 44 | 45 | 46 | 47 | 48 | 49 |
| 50 | 51 | 52 | 53 | 54 | 55 | 56 | 57 | 58 | 59 |
| 60 | 61 | 62 | 63 | 64 | 65 | 66 | 67 | 68 | 69 |
| 70 | 71 | 72 | 73 | 74 | 75 | 76 | 77 | 78 | 79 |
| 80 | 81 | 82 | 83 | 84 | 85 | 86 | 87 | 88 | 89 |
| 90 | 91 | 92 | 93 | 94 | 95 | 96 | 97 | 98 | 99 |
| 100 | 101 | 102 | 103 | 104 | 105 | 106 | 107 | 108 | 109 |
| 110 | 111 | 112 | 113 | 114 | 115 | 116 | 117 | 118 | 119 |
| 120 | 121 | 122 | 123 | 124 | 125 | 126 | 127 | 128 | 129 |
| 130 | 131 | 132 | 133 | 134 | 135 | 136 | 137 | 138 | 139 |
| 140 | 141 | 142 | 143 | 144 | 145 | 146 | 147 | 148 | 149 |
| 150 | 151 | 152 | 153 | 154 | 155 | 156 | 157 | 158 | 159 |
| 160 | 161 | 162 | 163 | 164 | 165 | 166 | 167 | 168 | 169 |
| 170 | 171 | 172 | 173 | 174 | 175 | 176 | 177 | 178 | 179 |
| 180 | 181 | 182 | 183 | 184 | 185 | 186 | 187 | 188 | 189 |
| 190 | 191 | 192 | 193 | 194 | 195 | 196 | 197 | 198 | 199 |
| 200 | 201 | 202 | 203 | 204 | 205 | 206 | 207 | 208 | 209 |
| 210 | 211 | 212 | 213 | 214 | 215 | 216 | 217 | 218 | 219 |
| 220 | 221 | 222 | 223 | 224 | 225 | 226 | 227 | 228 | 229 |
| 230 | 231 | 232 | 233 | 234 | 235 | 236 | 237 | 238 | 239 |
| 240 | | | | | | | | | |

In the preferred embodiments, the particular molecular weight gives advantages to the composition properties. With the preferred molecular weight, the chitin derivative is less vulnerable to the Maillard reaction, which can change the composition of the chitin derivative during the drying process, thus leading to reduced efficacy. Another advantage of the preferred molecular weight for the chitin derivative is the reduced amount of enzyme used and shorter reaction time. Yet, other advantages to the chitin derivative of the preferred embodiment are a reduced antimicrobial effect and a higher yield of chitin derivative as compared to chitin derivatives having a molecular weight lower than 10 kDa.

The following advantages are observed with increasing value of molecular weight of the chitin derivative.

a) reduction of reaction time or quantity of enzyme;

b) higher yield;

c) reduced antimicrobial effect; and

d) reduced susceptibility to the Maillard reaction.

### Reduction of Reaction Time or Quantity of Enzyme

A chitin derivative having a higher molecular weight would require using less enzyme or a shorter time for hydrolysis, and would thus generate many benefits. For instance, in a certain experimental condition of industrial production, the amount of time required to obtain a chitin derivative with a molecular weight of about 30 kDa is 170 minutes. In order to obtain a chitin derivative with a molecular weight of 40 kDa, the time required for hydrolysis is reduced by about 30%. The amount of enzyme required to obtain a chitin derivative having a molecular weight of 40 kDa is also lower. Therefore, there is a cut in the manufacturing cost of a chitin derivative having a higher molecular weight attributable either to a reduction of reaction time or by the use of a smaller quantity of enzymes required for hydrolysis.

### Higher Yield

The yield from the precipitation of a chitin derivative with molecular weights of at least 10 kDa is higher than that of a chitin derivative with molecular weights ranging lower than 10 kDa. Previous results obtained by the inventors indicate differences of 5% at 4° C. and of 10% at room temperature respectively (see WO 2005/066213). Therefore, there is a possibility of cutting the manufacturing cost by using a chitin derivative with a molecular weight equal to or greater than 10 kDa.

### Reduced Antimicrobial Effect

The antimicrobial effect would be less pronounced by using a chitin derivative with a molecular weight greater than 10 kDa; indeed, as the size of the molecule diminishes, the antimicrobial effect becomes more pronounced. Experiments in the inventors laboratory demonstrated that the antimicrobial effect of chitin derivative against E. coli, a dominant bacterium in the intestinal microflora, is at a maximum when using a chitin derivative with a molecular weight ranging between 8 and 15 kDa and decreases as the inventors used a chitin derivative with molecular weight away from those used within the previous range. As the chitin derivative of the preferred embodiments is taken during long periods of time in a continuous fashion, day after day, the small daily effect is amplified over several weeks, and even over several months. Therefore, it is believed that the chitin derivative of the preferred embodiments should interfere less with the intestinal flora.

### Reduced Susceptibility to the Maillard Reaction

As one skilled in the art would understand, if a chitosan with a molecular weight of 500,000 Daltons (g/mole) is hydrolyzed to 40,000 Daltons, (500,000 g/mole)/(40,000 g/mole)=12.5 divisions or 12.5 reducing units will be generated. However, if this same chitosan is hydrolized to 30,000 Daltons, (500,000 g/mole/(30,000 g/mole)=16.7 divisions or 16.7 reducing units will be generated. Therefore, further hydrolysis produces more reducing groups (33.6% in the above mentioned example) and enhances susceptibility to the Maillard reaction. By conducting tests on rats, the inventors have demonstrated that a chitosan modified by the Maillard reaction (resulting in a brown coloration) loses its hypocholesterolemic efficacy. Thus, a chitin derivative of a higher molecular weight should undergo the Maillard reaction to a lesser extent than that of a lower molecular weight during the drying or the atomisation process. In principle, the manufacturing process developed by the inventors minimises the Maillard reaction during atomisation in order to obtain a product which is as white as possible. However, deviations from the optimal parameters are always possible in a large scale routine production line. The preferred chitin derivative of the preferred embodiments would therefore be less susceptible of becoming brown in these sub-optimal conditions and would thus conserve a higher proportion of its hypocholesterolemic activity.

### Deacetylation

Chitin can be deacetylated partially or totally. Naturally occurring chitin is acetylated, with about 16% deacetylation. Chitosan refers to chitin that has been partially or fully deacetylated. Chitosan is a polysaccharide formed primarily of repeating units of β (1-4) 2-amino-2-deoxy-D-glucose (or D-glucosamine). Further deacetylation of chitin can be achieved by processing of chitin. Deacetylation values can vary with chitin sources and with processing methods.

Since chitosan is made by deacetylation of chitin, the term degree of deacetylation (DAC) can be used to characterize chitosan. This value gives the proportion of monomeric units of which the acetylic groups that have been removed, indicating the proportion of free amino groups (reactive after dissolution in weak acid) on the polymer. DAC could vary from about 70 to about 100%, depending of the manufacturing method used. This parameter indicates the cationic charge of the molecule after dissolution in a weak acid. There are many methods of DAC measurements, such as UV and infrared spectroscopy, acid-base titration, nuclear magnetic resonance, dye absorption, and the like. Since there are no official standard methods, numbers tend to be different for different methods. In high value product, NMR can give a precise DAC number. However, titration or dye adsorption can serve as a quick and convenient method and yield similar results as NMR.

Chitin deacetylation towards chitosan can be obtained by various methods. The most used method is that of alkaline treatment (Horowitz, S. T. et al., 1957). With this method, around 80% of deacetylation can be achieved without significant decrease of molecular weight. A more intense deacetylation cannot be obtained by this method without a simultaneous uncontrolled decrease of the degree of polymerization. A more promising method is deacetylation by a thermo-mechano-chemical treatment (Pelletier et al., 1990). This method allows a more careful control of the various characteristics of the final product (average degree of polymerisation and of deacetylation). Finally, a third method (Domard and Rinaudo, 1983) allows obtainment of a totally deacetylated product.

In a certain deacetylation protocol, when chitin is heated in a basic solution, such as a strong solution of sodium hydroxide (such as >about 40%) at high temperature (such as about 90-120° C.), chitosan is formed by deacetylation. This treatment can remove acetylic grouping on the amine radicals to a product (chitosan) that could be dissolved. It is said that at least 65% of the acetylic groups should be removed on each monomeric chitin to obtain the ability of being put in solution. The degree of deacetylation will vary according to the treatment conditions, such as duration, the temperature, and the concentration of the basic solution.

In the preferred embodiments, the chitin derivative has a deacetylation higher than about 80%. Preferably, the chitin derivative has a deacetylation higher than about 89%. More preferably, the chitin derivative has a deacetylation higher than about 89, 90, 91, 92, 93, 94, 95, 96, 97, 98, 99, or 100%. In a chitin derivative that has been deacetylated about 100%, the advantage being the chitin derivative forms a relatively homogeneous composition.

### Pharmaceutical Compositions

The compounds useful in the preferred embodiments can be presented with an acceptable carrier in the form of a pharmaceutical composition. The carrier is acceptable in the sense of being compatible with the other ingredients of the composition and is not deleterious to the recipient. The carrier can be a solid or a liquid, or both, and is preferably formulated with the compound as a unit-dose composition, for example, a capsule or tablet, which can contain from about 0.05% to about 95% by weight of the active compound. Examples of suitable carriers, diluents, and excipients include, but are not limited to, lactose, dextrose, sucrose, sorbitol, mannitol, starches, gum acacia, alginates, tragacanth, gelatin, calcium silicate, cellulose, magnesium carbonate, or a phospholipid with which the polymer can form a micelle. Other pharmacologically active substances can also be present. The pharmaceutical compositions of the preferred embodiments can be prepared by any of the well-known techniques of pharmacy, comprising admixing the components.

As previously mentioned, the use of chitosan derivative allows the production of a pharmaceutical composition that may have a prolonged shelf life compared to the use of natural chitosan. It is a well-established fact that uncharged primary amines are more susceptible to oxidation. In contrast, the corresponding salts confer increased stability due to the fact of protonation of the lone pair of electrons of the nitrogen atom. This basic principle also applies to the chitosan polymer due to the presence of the large number of primary amino groups (D-glucosamine units) composing its backbone. In this respect, the salts of chitosan described above will confer stability over long periods of storage.

Whereas a number of salts of chitosan can be used to increase its stability under storage conditions, the selection of the nature of the chitosan salt may be dictated by the intended purpose of its use. For instance, chitosan salts that are compatible with food offer a definitive advantage for their uses as a diet supplement or for other purposes related to human or animal applications. The citrate salt of chitosan has been found to fulfill this requirement in two ways. First, it is a food-compatible salt and second, it confers to the natural chitosan molecule an extended shelf life.

In practicing the methods of the preferred embodiments, administration of the preferred embodiments may be accomplished by oral route, or by intravenous, intramuscular, subcutaneous injections, or a combination thereof.

For oral administration, preferred embodiments can be in the form of, for example, but not limited to, a tablet, a capsule, a suspension, powders (e.g., for sprinkling on food), or liquid. The liquid product formulation may also encompass a colloid/emulsion in water or solvent such as a solvent or an oil. Capsules, tablets, liquid, or powders, and the like can be prepared by conventional methods well-known in the art. The compounds are preferably made in the form of a dosage unit containing a specified amount of the compound. In one embodiment, the composition is in the form of a sustained release formulation.

When the chitosan derivative in the form of powder is obtained, encapsulation proceeds. If the powder is composed of multiple batches, a “tri bender” is thus used to provide a uniform admixture of the various batches. In some cases, the powder granulometry is not uniform and a sieving of the powder is therefore necessary in order to obtain the required granulometry for the type of encapsulation equipment that is used. Such sieving of the powder is accomplished either by coring or gravity. During encapsulation, some capsules are sampled and weighed to provide a uniform filling. Capsules of size 00 are used to hold 800 mg of chitosamine derivative per capsule. Capsules of size 00 or 01 may also be used for lower chitosamine derivative doses, for example 400 mg to 600 mg.

A preferred total daily dose of about 400 mg to about 4.8 grams per day and preferably between about 800 mg and 3.2 grams per day may generally be appropriate. More preferably, the total daily dose may range from 1.6 grams to 2.4 grams per day. The chitin derivative will preferably be taken three times a day, or preferably twice a day and more preferably once a day in a sustained release system (mode). The chitin derivative will preferably be taken with meals.

The daily doses for the preferred embodiments can be administered to the patient in a single dose, or in proportionate multiple subdoses. Subdoses can be administered about 2 to about 6 times per day. Doses can be in sustained release form effective to obtain desired results.

The dosage regimen to treat hyperlipidemia and hyperlipidemia-associated conditions, and reduce plasma cholesterol with the preferred embodiments is selected in accordance with a variety of factors. These factors include, but are not limited to, the type, age, weight, sex, diet, and medical condition of the patient, the severity of the disease, the route of administration, pharmacological consideration, such as the activity, efficacy, pharmacokinetics and toxicology profiles of the particular compound employed, whether a drug delivery system is utilized, and whether the compound is administered as part of a drug combination. Thus, the dosage regimen actually employed may vary widely and therefore deviate from the preferred dosage regimen set forth above.

Initial treatment of a patient suffering from a hyperlipidemic condition, such as, but not limited to, hypercholesterolemia and atherosclerosis, can begin with the dosages indicated above. Treatment should generally be continued as necessary over a period of several weeks to several months or years until the condition has been controlled or eliminated. Patients undergoing treatment with the compounds or compositions disclosed herein can be routinely monitored by, for example, measuring serum LDL and total cholesterol levels by any of the methods well-known in the art, to determine the effectiveness of the therapy.

### Nutraceuticals

The chitin derivative useful in the preferred embodiment can be incorporated in a functional food or nutraceutical. This compound may be presented in the form of active agents such as cholesterol lowering agents. As such, this compound may be useful in the manufacture of nutraceuticals and/or functional foods useful for preventing hyperlipidemia associated conditions.

In a preferred embodiment, the chitin derivative compound is incorporated in functional foods including but not limited to: beverages, including but not limited to sodas, water, sports/energy drinks, canned and bottled juices, fresh and refrigerated juices, frozen juices, yoghurt drinks, smoothies, teas and coffees; breads and grains, including but not limited to breakfast cereals, breads, baked goods, baking ingredients such as flour, frozen breads, dried breads and crackers, pastas; snack foods, including but not limited to nutrition bars, weight loss bars, energy/sports bars, candy bars, chips, gum; packaged and prepared foods, including but not limited to frozen foods such as pizzas and dinners, canned and dried soups, desserts including cookies; condiments, including but not limited to dressings, spreads, sauces; dairy and dairy alternatives, including but not limited to milk, cheese, butter, ice cream, yoghurt, margarine and soymilk.

According to another embodiment, the chitin derivative may be in the form of a dietary supplement or an over the counter medicine (OTC). Thus, the invention also concerns a functional food or dietary supplement, comprising an effective amount of a chitin derivative.

### Prevention and Treatment of Conditions

The preferred embodiments can be used to prevent, give relief from, or ameliorate a disease condition having hyperlipidemia as an element of a disease, such as atherosclerosis or coronary heart disease, or to protect against or treat further high cholesterol plasma or blood levels with the compounds and/or compositions of the preferred embodiments. The pharmaceutical composition of the preferred embodiments thus prevents, gives relief from or ameliorates the above-mentioned hyperlipidemia-associated diseases by increasing the level of HDL, decreasing the level of LDL and/or decreasing the level of total cholesterol by increasing the ratio of HDL/LDL. Hyperlipidemia is an elevation of lipids (fats) in the bloodstream. These lipids include cholesterol (including HDL, LDL), cholesterol esters (compounds), phospholipids, triglycerides, and fatty acids. These lipids are transported in the blood as part of large molecules called lipoproteins.

Adverse effects of hyperlipidemia include atherosclerosis and coronary heart disease. Atherosclerosis is a disease characterized by the deposition of lipids, including cholesterol, in the arterial vessel wall, resulting in a narrowing of the vessel passages and ultimately hardening the vascular system. The primary cause of coronary heart disease (CHD) is atherosclerosis. CHD occurs when the arteries that supply blood to the heart muscle (coronary arteries) become hardened and narrowed. As a result of CHD, there could be angina or heart attack. Over time, CAD can weaken your heart muscle and contribute to heart failure or arrhythmias.

Hypercholesterolemia is also linked with cardiovascular disease. Cardiovascular disease refers to diseases of the heart and diseases of the blood vessel system (arteries, capillaries, veins) within a person's entire body, such as the brain, legs, and lungs. Cardiovascular diseases include, but are not limited to, coronary heart disease, peripheral vascular disease, and stroke.

Accordingly, the preferred embodiments may be used in preventing or treating hyperlipidemia and conditions associated with hyperlipidemia, such as hypercholesterolemia, atherosclerosis, coronary heart disease, and cardiovascular disease.

The preferred embodiments also have a lower antibacterial effect, therefore having fewer side effects. The preferred embodiments possess a molecular weight such that they are less disruptive to intestinal flora. The gut serves as the natural habitat for a great number of bacteria—some beneficial to the host, others harmful. One of the more common side effect of an antibacterial composition is diarrhea, which results from the composition disrupting the balance of intestinal flora.

### Example

Studies done by the inventor have shown the cholesterol-lowering efficacy of the chitin derivatives of the preferred embodiment.

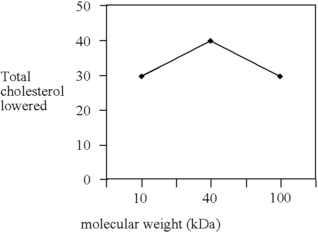

The disclosure below is of specific examples setting forth preferred methods. These examples are not intended to limit the scope, but rather to exemplify preferred embodiments.

What is claimed is:

1. A pharmaceutical composition comprising chitin derivative having a molecular weight of at least 10 kDa.

2. The pharmaceutical composition of claim 1, wherein the chitin derivative has a molecular weight of at least 10 kDa to about 240 kDa.

3. The pharmaceutical composition of claim 2, wherein the chitin derivative has a molecular weight of at least 30 kDa to about 80 kDa.

4. The pharmaceutical composition of claim 3, wherein the chitin derivative has a molecular weight of at least 40 kDa to about 70 kDa.

5. The pharmaceutical composition of claim 1, wherein the chitin derivative is further deacetylated by chemical or biological treatment.

6. The pharmaceutical composition of claim 5, wherein the chitin derivative is deacetylated at least about 80%.

7. The pharmaceutical composition of claim 5, wherein the chitin derivative is deacetylated at least about 89%.

8. The pharmaceutical composition of claim 5, wherein the chitin derivative is deacetylated at about 93%.

9. The pharmaceutical composition of claim 1, wherein the chitin derivative comprises a negatively charged anion.

10. The pharmaceutical composition according to claim 9, wherein the anion is an organic or inorganic anion.

11. The pharmaceutical composition according to claim 10, wherein the anion is an organic anion selected from the group consisting of malate, tartrate, citrate, lactate, succinate and amino acids.

12. The pharmaceutical composition of claim 11, wherein the organic anion is citrate.

13. The pharmaceutical composition of claim 10, wherein the anion is an inorganic anion selected from the group consisting of sulfate, phosphate, chloride and thiosulfate.

14. The pharmaceutical composition of claim 13, wherein the inorganic anion is phosphate.

15. The pharmaceutical composition of claim 9, wherein the anion is an antioxidant.

16. The pharmaceutical composition of claim 9, wherein the anion is a polymeric anion.

17. The pharmaceutical composition of claim 1, for the prevention or treatment of hyperlipidemia or hyperlipidemia associated conditions.

18. The pharmaceutical composition of claim 17, wherein the hyperlipidemia associated conditions are selected from the group consisting of hypercholesterolemia, atherosclerosis, coronary heart disease and cardiovascular disease.

19. The pharmaceutical composition of claim 1, further comprising a pharmaceutically acceptable carrier.

20. A method for the prevention or treatment of hyperlipidemia or hyperlipidemia-associated conditions comprising administering a pharmaceutical composition of claim 1.

21. The method of claim 20, wherein the hyperlipidemia-associated condition is selected from the group consisting of hypercholesterolemia, atherosclerosis, coronary heart disease, and cardiovascular disease.

22. The method of claim 20, wherein the chitin derivative has a molecular weight of at least 10 to about 240 kDa.

23. The method of claim 20, wherein the chitin derivative is further deacetylated by chemical or biological treatment.

24. The method of claim 23, wherein the chitin derivative is deacetylated at least 80%.

25. The method of claim 23, wherein the chitin derivative is deacetylated at least 89%.

26. The method of claim 23, wherein the chitin derivative is deacetylated 100%.

27. A chitin derivative having a molecular weight of at least 10 to about 240 kDa.

28. The chitin derivative of claim 27 having a molecular weight of at least 30 to about 80 kDa.

29. The chitin of claim 27, having a molecular weight of about 40 to about 70 kDa.

30. The chitin of claim 27, being deacetylated at least about 80%.

31. The chitin of claim 27, being deacetylated at least about 89%.

32. The chitin of claim 27, being deacetylated at about 93%.

33. The chitin derivative of claim 27, comprising a negatively charged anion.

34. The chitin derivative of claim 33, wherein the anion is an organic or inorganic anion.

35. The chitin derivative of claim 34, wherein the anion is an organic anion selected from the group consisting of malate, tartrate, citrate, lactate, succinate and amino acids.

36. The chitin derivative of claim 35, wherein the organic anion is citrate.

37. The chitin derivative of claim 34, wherein the anion is an inorganic anion selected from the group consisting of sulfate, phosphate, chloride and thiosulfate.

38. The chitin derivative of claim 37, wherein the inorganic anion is phosphate.

39. The chitin derivative according to claim 33, wherein the anion is an antioxidant.

40. The chitin derivative according to claim 33, wherein the anion is a polymeric anion.

41. A functional food comprising a chitin derivative as defined in claim 27.

42. The functional food of claim 41, selected from the group consisting of beverages, breads, grains, snack foods, packaged and prepared foods, condiments, dairy and dairy alternatives.
